(19) United States
(12) Patent Application Publication
(10) Pub. No.: US 2024/0027424 A1
(43) Pub. Date: Jan. 25, 2024

(54) SAMPLE PREPARATION DEVICE AND SAMPLE PREPARATION SYSTEM

(71) Applicant: SONY GROUP CORPORATION, TOKYO (JP)

(72) Inventors: HIDEYA CHUBACHI, TOKYO (JP); KENZO MACHIDA, TOKYO (JP); YOSHIAKI KATO, TOKYO (JP); AYA FUCHIGAMI, TOKYO (JP)

(21) Appl. No.: 18/041,430

(22) PCT Filed: Jul. 15, 2021
(86) PCT No.: PCT/JP2021/026594

§ 371 Date: Feb. 13, 2023

(30) Foreign Application Priority Data
Aug. 25, 2020 (JP) 2020-141546

(51) Int. Cl. G01N 33/49; B01L 3/00; G01N 1/34

(52) U.S. Cl. CPC G01N 33/491; B01L 3/502; G01N 1/34; B01L 2300/0883; B01L 2400/0409; B01L 2300/0858; B01L 2300/0681; B01L 2200/0647; B01L 2200/0631

(57) ABSTRACT

An object of the present technology is to provide a sample preparation system for increasing a content ratio of target cells. The present technology provides a sample preparation device including a container, and a channel in which a bioparticle-containing liquid flows, the channel accommodated in the container, in which the channel is formed such that a centrifugal force acts on the bioparticle-containing liquid, and an outer peripheral wall of the channel is formed such that at least part of components of the bioparticle-containing liquid may be transferred to an outside of the channel. Furthermore, the present technology also provides a sample preparation system including the sample preparation device, and an analysis device that executes analysis of the bioparticle-containing liquid that passes through the channel.

### TECHNICAL FIELD

The present technology relates to a sample preparation device and a sample preparation system, and particularly relates to a sample preparation device and a sample preparation system used for preparing a sample containing bioparticles.

### BACKGROUND ART

In order to analyze blood cells, bioparticle analysis such as flow cytometry (hereinafter also referred to as FCM) is performed. Since blood contains many kinds of components, it is desirable that a sample subjected to the bioparticle analysis does not contain components that are not to be analyzed as much as possible.

Regarding a technology of excluding components that are not to be analyzed, for example, Patent Document 1 below discloses “A blood processing device including: a centrifuge rotor; a separation chamber attached to the centrifuge, the separation chamber including an outflow line in which at least a portion of the outflow line extends from the centrifuge rotor; a solution line in fluid communication with the at least one outflow line; and a collection chamber including an inlet and an outlet, in which the outlet of the separation chamber is in fluid communication with the inlet of the collection chamber.”

### CITATION LIST

### Patent Document

Patent Document 1: Japanese Unexamined Patent Publication No. 2013-514863

### SUMMARY OF THE INVENTION

### Problems to be Solved by the Invention

A sample to be subjected to bioparticle analysis may be subjected to a process of increasing a ratio of bioparticles to be analyzed. An object of the present technology is to provide a new method for simply and efficiently performing the process.

### Solutions to Problems

The present inventors found that the above-described problem may be solved by a specific sample preparation device.

That is, the present technology provides
a sample preparation device including:
a container; and
a channel in which a bioparticle-containing liquid flows, the channel accommodated in the container, in which
the channel is formed such that a centrifugal force acts on the bioparticle-containing liquid, and
an outer peripheral wall of the channel is formed such that at least part of components of the bioparticle-containing liquid may be transferred to an outside of the channel.

In one embodiment of the present technology, the channel may have a spiral shape.

In this embodiment, the channel may have a curved shape so as to go around one axis.

In this embodiment, the channel may be formed so as to go around the axis one or more times.

In this embodiment, the outer peripheral wall of the channel may have a predetermined curvature.

In another embodiment of the present technology, the channel may have a cylindrical shape.

In this embodiment, the bioparticle-containing liquid may be formed to form a flow that goes around the axis of the cylindrical shape.

In still another embodiment of the present technology, the channel may have a U shape.

In this embodiment, a plurality of the channels having the U-shape may be included, and the plurality of U-shaped channels may be connected to each other to form a single line of flow.

In the present technology, the outer peripheral wall may be porous.

It is possible that the outer peripheral wall allows part of bioparticles contained in the bioparticle-containing liquid to pass and does not allow remaining bioparticles to pass.

The container may include a first inlet that introduces the bioparticle-containing liquid into the channel, and a first outlet that discharges the bioparticle-containing liquid that passes through the channel to an outside of the container, and

the container may include a second inlet that introduces a liquid that receives the components transferred to the outside of the channel into the container, and a second outlet that discharges the liquid to the outside of the container.

The sample preparation device may be formed such that the liquid introduced from the second inlet swirls to flow in the container.

The second inlet and the second outlet may open toward a position deviated from a central axis of the container.

The second inlet may be arranged above the second outlet.

The container may include a plurality of second inlets and a plurality of second outlets.

The sample preparation device of the present technology may include a plurality of sets of the container and the channel, in which

sizes of components that may be transferred to the outside from the outer peripheral wall of the channel of respective sets may be different from each other.

The sample preparation device of the present technology may be formed such that the bioparticle-containing liquid output from the first outlet may enter the container again from the first inlet.

The sample preparation device of the present technology may be used for separating blood components.

Furthermore, the present technology also provides
a sample preparation system including:
a sample preparation device including a container, and a channel in which a bioparticle-containing liquid flows, the channel accommodated in the container, the channel formed such that a centrifugal force acts on the bioparticle-containing liquid, and an outer peripheral wall of the channel formed such that components of the bioparticle-containing liquid may be transferred to an outside of the channel; and
an analysis device that executes analysis of the bioparticle-containing liquid that passes through the channel.

Furthermore, the present technology also provides
a sample preparation device including:
a container; and
a channel in which a microparticle-containing liquid flows, the channel accommodated in the container, in which
the channel is formed such that a centrifugal force acts on the microparticle-containing liquid, and
an outer peripheral wall of the channel is formed such that at least part of components of the microparticle-containing liquid may be transferred to an outside of the channel.

Furthermore, the present technology also provides
a sample preparation system including:
a sample preparation device including a container, and a channel in which a microparticle-containing liquid flows, the channel accommodated in the container, the channel formed such that a centrifugal force acts on the microparticle-containing liquid, and an outer peripheral wall of the channel formed such that components of the microparticle-containing liquid may be transferred to an outside of the channel; and
an analysis device that executes analysis of the microparticle-containing liquid that passes through the channel.

### BRIEF DESCRIPTION OF DRAWINGS

FIG. 1 is a schematic diagram illustrating four layers formed by centrifuging whole blood using a Ficoll reagent.

FIG. 2 is a schematic diagram for explaining dead end filtration.

FIG. 3 is a schematic diagram for explaining cross-flow filtration.

FIG. 4 is a schematic diagram illustrating a configuration example of a sample preparation device of the present technology.

FIG. 5 is a schematic diagram of a configuration example of a sample preparation system including the sample preparation device of the present technology.

FIG. 6 is an example of a flowchart of a sample preparation method using the sample preparation device of the present technology.

FIG. 7 is a schematic diagram illustrating a configuration example of a sample preparation device of the present technology.

FIG. 8 is a schematic diagram illustrating a channel included in a sample preparation device of the present technology.

FIG. 9 is a schematic diagram illustrating the channel included in the sample preparation device of the present technology.

FIG. 10 is a schematic diagram illustrating a configuration example of a sample preparation device of the present technology.

FIG. 11 is a schematic diagram illustrating a channel included in a sample preparation device of the present technology.

### MODE FOR CARRYING OUT THE INVENTION

A preferred mode for carrying out the present technology is hereinafter described. Note that, embodiments hereinafter described are representative embodiments of the present technology, and the scope of the present technology is not limited only to them. Note that, the present technology is described in the following order.

1. First Embodiment (Sample Preparation Device)

(1) Description of First Embodiment

(2) Example of Sample Preparation Device according to Present Technology (Channel Having Spiral Shape)

(2-1) Configuration Example of Sample Preparation Device

(2-2) Example of System Including Sample Preparation Device and Example of Sample Preparation Method

(2-2-1) Configuration Example of Sample Preparation System

(2-2-2) Sample Preparation Method

(2-2-3) Operation Example (Example of Preparing Sample Having High Content Ratio of White Blood Cells (WBCs) by Removing Red Blood Cells (RBC) from Blood)

(3) First Variation of Sample Preparation Device according to Present Technology (Channel Having Cylindrical Shape)

(4) Second Variation of Sample Preparation Device according to Present Technology (Channel Having U Shape)

(5) Third Variation of Sample Preparation Device according to Present Technology (Connection of Plurality of Devices)

(6) Fourth Variation of Sample Preparation Device according to Present Technology (Density Adjustment)

2. Second Embodiment (Sample Preparation System)

### 1. First Embodiment (Sample Preparation Device)

(1) Description of First Embodiment

A sample preparation device of the present technology includes a container, and a channel in which a bioparticle-containing liquid flows accommodated in the container. The channel is formed such that a centrifugal force acts on the bioparticle-containing liquid, and an outer peripheral wall of the channel is formed such that at least part of components of the bioparticle-containing liquid may be transferred to the outside of the channel. Therefore, while bioparticles flow in the channel, the at least part of components may be transferred to the outside of the channel by the action of the centrifugal force. Therefore, it is possible to separate unintended components from target bioparticles, and it is possible to easily and efficiently increase a ratio of the target bioparticles.

The present technology will be hereinafter described in further detail with reference to the conventional technology.

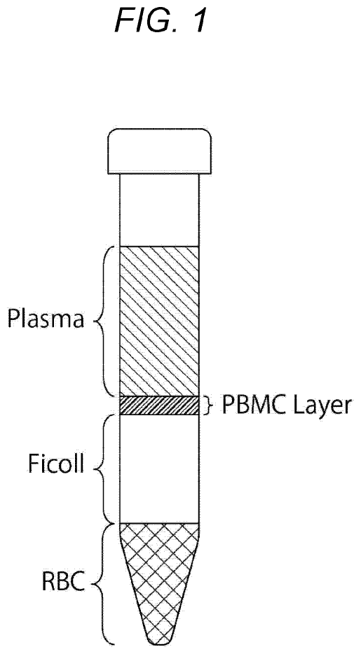

A process of separating the target bioparticles from the unintended components is often performed for analyzing blood cells. In order to analyze the blood cells, for example, peripheral blood mononuclear cells (hereinafter also referred to as “PBMCs”) are separated from red blood cells (hereinafter also referred to as “RBCs”). As a method for separating the PBMCs from the RBCs to recover, a method of performing density gradient centrifugation using a Ficoll reagent is known. The Ficoll reagent used in this method has intermediate density (specific gravity) between that of the PBMCs and RBCs, and by adding the reagent to the blood and performing the centrifugation, a space is formed between the PBMCs and RBCs by a Ficoll reagent layer. For example, by putting whole blood to which the Ficoll reagent is added into a tube as illustrated in FIG. 1 and performing centrifugation, the whole blood is divided into four layers of plasma, a PBMC layer, the Ficoll reagent, and the RBCs as illustrated in FIG. 1. Then, by collecting only the PBMC layer with a pipette, the PBMCs separated from the RBCs are obtained.

However, since the collection with a pipette is performed manually, it is difficult to collect all the PBMCs as the target cells, and a recovery rate is low. Furthermore, since the collection with a pipette is performed manually, there also is a case where the RBC is sucked up. Therefore, in order to increase the recovery rate of the PBMCs, experience and skill are required. Moreover, in this method, a manual operation other than the collection with a pipette also needs to be performed, which is complicated.

Furthermore, in this method, since the centrifugation is usually performed using, for example, a 15 ml tube, a 50 ml tube or the like, it is difficult to process a large amount of samples.

Furthermore, since this method is performed in an open system, an aseptic operation cannot be performed.

An improved method of the above-described method is also developed. For example, a tube pre-filled with special gel or filter in addition to a reagent having a predetermined specific gravity such as the Ficoll reagent is commercially available (such as, for example, BD Vacutainer (registered trademark) CPT (trademark) Mononuclear Cell Preparation Tube and Lymphoprep (trademark) Tube). However, even in a case where these tubes are used, manual PBMC collection with a pipette is still necessary, the recovery rate is low, and experience and skill are required to increase the recovery rate. Moreover, also in a case of using these tubes, a manual operation other than the collection with a pipette needs to be performed, which is complicated. Furthermore, it is difficult to process a large amount of samples with these tubes. Furthermore, since the process using these tubes is performed in an open system, an aseptic operation cannot be performed.

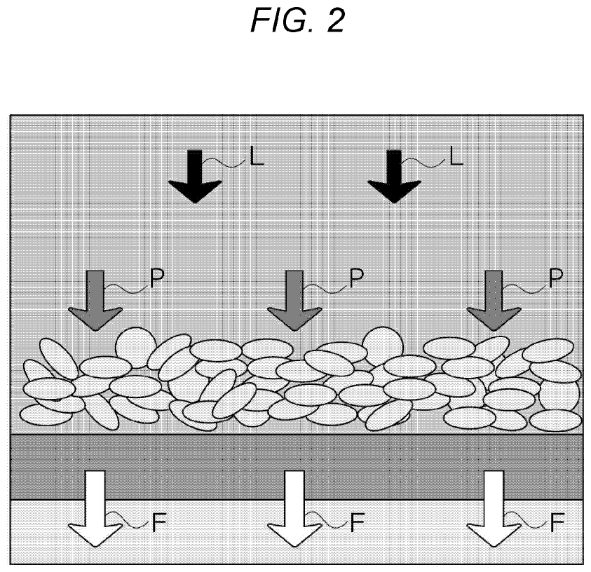

In order to separate the target cells from the unintended components, for example, it is conceivable to employ dead end filtration. In the dead end filtration, by using a filter having a hole diameter smaller than the target cell, the target cells are trapped by the filter. In order to recover the trapped cells, some solution is allowed to flow in an opposite direction to recover. In the dead end filtration, for example, as illustrated in FIG. 2, a flow L of a sample is formed in a direction perpendicular to a surface of the filter, and a pressure P is applied in the same direction. Therefore, filtrate F that passes through the filter is generated, and accordingly, unintended cells pass through the hole. In contrast, the target cells are trapped by the filter.

In order to recover the cells trapped by the filter by the dead end filtration, it is necessary to reverse the solution. However, even when this is reversed, it is difficult to completely recover the trapped cells, and a certain number of cells remain on the filter. Therefore, the recovery rate of the target cells by the dead end filtration is low. Furthermore, in the dead end filtration, the filter is easily clogged and cannot be applied to a thick solution.

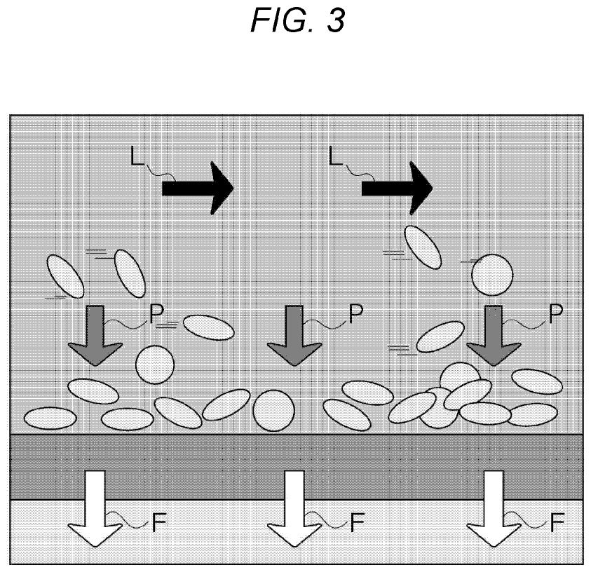

In order to separate the target cells from the unintended cells, it is also conceivable to employ cross-flow filtration (also referred to as tangential flow). In the cross-flow filtration, a tube (membrane) with holes on a side face, particularly a hollow fiber is used. In the cross-flow filtration, a solution is allowed to flow in the hollow fiber, and a pressure inside the tube is made higher than a pressure outside the tube, so that filtrate that flows out of the tube is generated. For example, as illustrated in FIG. 3, a flow L of the solution is formed in a direction parallel to a wall surface of the hollow fiber, and a pressure P is applied in a direction perpendicular to a tube wall surface. Therefore, filtrate F that passes through the tube wall surface is formed.

However, the hollow fiber used in the cross-flow filtration has a limited hole diameter of about 0.65 μm at the maximum. Therefore, cells having a larger size than this cannot be separated. Furthermore, in the cross-flow filtration, it is necessary to adjust the pressure inside the tube and the pressure outside the tube, and this adjustment might be difficult in some cases.

Furthermore, a hemolysis process is also known as a method for recovering only white blood cells from the whole blood. When the whole blood is centrifuged as is, red blood cells are usually accumulated at the bottom. In contrast, when the whole blood is centrifuged after the red blood cells are ruptured by addition of a hemolysis reagent to the whole blood, the white blood cells are accumulated at the bottom. Then, the red blood cells are removed by suction of supernatant.

However, the hemolysis reagent deteriorates viability of the cells that are wanted to be recovered. Furthermore, in this method, the supernatant is removed manually with a pipette. Even when only the supernatant is wanted to be sucked, the white blood cells might be sucked to some extent, and the recovery rate is poor. Experience and skill are required to increase the recovery rate. Moreover, this method involves a lot of manual operations and is complicated. Furthermore, in this method, since centrifugation is usually performed using, for example, a 15 ml tube, a 50 ml tube or the like, it is difficult to process a large amount of samples. Furthermore, since this method is performed in an open system, an aseptic operation cannot be performed.

By performing an operation of allowing the bioparticle-containing liquid to flow in the channel included in the sample preparation device of the present technology, it is possible to separate the unintended components from the target bioparticles. Therefore, in the present technology, a complicated manual operation required in the above-described method of performing the centrifugation is unnecessary, and the ratio of the target bioparticles may be easily increased.

Furthermore, in the sample preparation device of the present technology, the operation of allowing the bioparticle-containing liquid to flow in the channel is performed, the bioparticles remaining on the channel wall surface may be reduced, so that the recovery rate may be increased.

Furthermore, the sample preparation device of the present technology may process a large amount of samples unlike the method using the tube and the like described above.

Furthermore, in the present technology, the pressure applied to the bioparticles is smaller than that in the dead end filtration. Therefore, damage to the recovered bioparticles may be suppressed, and for example, the viability of the recovered cells is improved.

Furthermore, in the present technology, it is not required to perform pressure adjustment necessary for the cross-flow filtration. Therefore, the target bioparticles may be easily recovered.

In one embodiment of the present technology, the channel may have a spiral shape. When a bioparticle-containing liquid is allowed to flow in the channel having the spiral shape, a centrifugal force acts on the liquid. Then, at least part of components of the bioparticle-containing liquid are transferred to the outside of the channel by the centrifugal force. This embodiment is described in further detail in (2) below.

In another embodiment of the present technology, the channel may have a cylindrical shape. When a bioparticle-containing liquid is allowed to flow in the channel having the cylindrical shape also, a centrifugal force may act on the liquid. Then, at least part of components of the bioparticle-containing liquid are transferred to the outside of the channel by the centrifugal force. This embodiment is described in further detail in (3) below.

In still another embodiment of the present technology, the channel may have a U shape. When a bioparticle-containing liquid is allowed to flow in the channel having the U shape also, a centrifugal force may act on the liquid. Then, at least part of components of the bioparticle-containing liquid are transferred to the outside of the channel by the centrifugal force. This embodiment is described in further detail in (4) below.

In the present technology, the container includes a first inlet that introduces the bioparticle-containing liquid into the channel, and a first outlet that discharges the bioparticle-containing liquid that passes through the channel to the outside of the container, and the container includes a second inlet that introduces a liquid that receives the component transferred to the outside of the channel into the container, and a second outlet that discharges the liquid out of the container.

By the second inlet and the second outlet, the liquid that receives the components output from the channel by the action of the centrifugal force may be supplied into the container and discharged from the container, so that unintended components may be efficiently discharged from the container, for example.

In this specification, the bioparticles may be biological particles, and may mean, for example, particles forming an organism. The bioparticles may be microparticles.

The bioparticles may be, for example, cells. The cells may include animal cells (such as hemocyte cells) and plant cells. The cells may particularly be blood cells or tissue cells. Examples of the blood cells may include, for example, white blood cells (for example, peripheral blood mononuclear cells), red blood cells, and platelets, and the blood cells particularly include the white blood cells. Examples of the white blood cells may include, for example, monocytes (macrophages), lymphocytes, neutrophils, basophils, and eosinophils. The cells may be, for example, floating cells such as T cells and B cells. The tissue cells may be, for example, adherent cultured cells, adherent cells separated from the tissue or the like. Furthermore, the cells may be tumor cells. The cells may be cultured or uncultured. The bioparticles may be cell aggregation such as, for example, spheroid and organoid.

The bioparticles may be non-cellular biocomponents, for example, extracellular vesicles, particularly exosomes, microvesicles or the like.

The bioparticles may be microorganisms or viruses. The microorganisms may include bacteria such as Escherichia coli, and fungi such as yeast. The viruses may be, for example, a DNA virus or an RNA virus, and may be a virus with or without an envelope.

The bioparticles may also include biological polymers such as nucleic acids, proteins, and complexes thereof. These biological polymers may be, for example, extracted from the cells or may be included in blood samples or other liquid samples.

Furthermore, in the sample preparation device according to the present technology, a liquid containing non-bioparticles may be introduced into the channel in place of the bioparticle-containing liquid. A material forming the non-bioparticles may be, for example, an organic or inorganic material, particularly an organic or inorganic polymer material, or may be a metal. The organic polymer material includes polystyrene, styrene/divinylbenzene, polymethyl methacrylate and the like, for example. The inorganic polymer material includes glass, silica, a magnetic material and the like. The non-bioparticles may be, for example, latex particles or gel particles.

That is, the present technology also provides a sample preparation device used for processing a liquid (microparticle-containing liquid) containing the microparticles including the bioparticles and non-bioparticles. That is, the sample preparation device may include a container, and a channel in which a microparticle-containing liquid flows accommodated in the container, the channel may be formed such that a centrifugal force acts on the microparticle-containing liquid, and an outer peripheral wall of the channel may be formed such that at least part of components of the microparticle-containing liquid may be transferred to the outside of the channel.

In this specification, the bioparticle-containing liquid may be a liquid obtained from an organism, for example, a body fluid. The body fluid may be blood, lymph, tissue fluid (for example, intertissue fluid, intercellular fluid, interstitial fluid and the like), or body cavity fluid (for example, serous cavity fluid, pleural effusion, ascites, pericardial fluid, cerebrospinal fluid (spinal fluid), joint fluid (synovial fluid), and the like). Furthermore, the bioparticle-containing liquid may be a liquid obtained from these body fluids. In one embodiment of the present technology, the bioparticle-containing liquid may be the blood. That is, the sample preparation device of the present technology is used for separating blood components.

(2) Example of Sample Preparation Device according to Present Technology (Channel Having Spiral Shape)

(2-1) Configuration Example of Sample Preparation Device

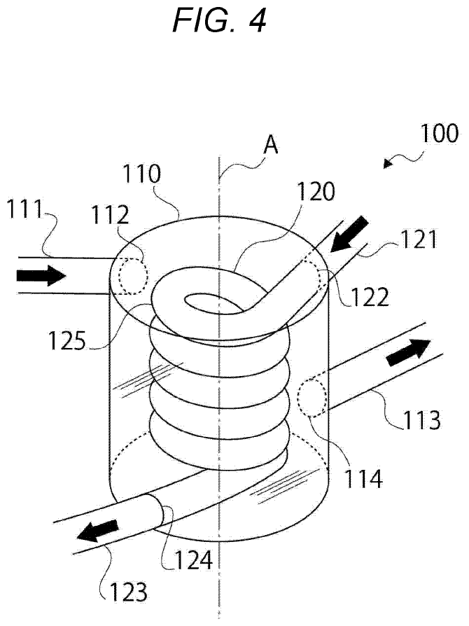

An example of a sample preparation device of the present technology is hereinafter described with reference to FIG. 4.

A sample preparation device 100 illustrated in FIG. 4 is provided with a container 110 and a channel 120 accommodated in the container 110. A bioparticle-containing liquid flows in the channel 120.

The container 110 only needs to be able to accommodate the channel 120, and a shape and a dimension thereof may be selected by those skilled in the art. The shape of the container 110 may be, for example, a cylindrical shape or a prismatic shape (for example, a quadrangular prism shape, a pentagonal prism shape, or a hexagonal prism shape). The shape of the container 110 is preferably a cylindrical shape. The cylindrical shape facilitates generation of a swirling flow in the container as described later. A diameter of the cylinder may be, for example, 3 cm or larger, 4 cm or larger, or 5 cm or larger. Furthermore, the diameter of the cylinder may be, for example, 50 cm or smaller, 40 cm or smaller, or 30 cm or smaller.

As illustrated in FIG. 4, the channel 120 has a spiral shape. When the bioparticle-containing liquid flows in the channel having the spiral shape, a centrifugal force acts on the bioparticle-containing liquid.

In this specification, the term of the spiral shape may mean a curved shape so as to go around one axis. For example, as illustrated in FIG. 4, the channel 120 has a curved shape so as to go around an axis A. Preferably, the channel 120 may be formed to go around the axis A one or more times, for example, two or more times, three or more times, or four or more times. Therefore, a section in which the centrifugal force acts on the bioparticle-containing liquid becomes longer, and an area in which the components of the bioparticle-containing liquid may be transferred to the outside of the channel may be increased. Therefore, unintended components may be efficiently transferred to the outside of the channel.

An upper limit value of the number of times the channel 120 goes around the axis A does not need to be particularly set, but may be determined according to factors such as a size of the container 110 and/or a size of the channel 120, for example. The number of times that the channel 120 goes around the axis A may be, for example, 100 times or smaller, 50 times or smaller, 20 times or smaller, or 10 times or smaller.

An outer peripheral wall 125 of the channel 120 is formed such that at least part of components (particularly at least part of bioparticles) of the bioparticle-containing liquid may be transferred to the outside of the channel. Therefore, when the centrifugal force acts on the bioparticle-containing liquid, the transfer of the at least part of components to the outside of the channel 120 is promoted, and the unintended components (for example, the unintended bioparticles) may be separated from the target bioparticles, for example. The outer peripheral wall 125 may be a wall of a portion with which the at least part of components on which the centrifugal force acts are brought into contact.

The outer peripheral wall 125 may have a predetermined curvature, for example. The curvature may be, for example, ⅕ [1/mm] to 1/50 [1/mm], and particularly 1/10 [1/mm] to 1/20 [1/mm].

Furthermore, the sample preparation device 100 may be formed such that relative centrifugal acceleration of, for example, 10 [G] to 1,000 [G], particularly 20 [G] to 8,000 [G] is applied to the bioparticle-containing liquid.

A radius of the spiral may be, for example, 5 [mm] or larger, 7 [mm] or larger, or 10 [mm] or larger. The radius of the spiral may be 50 [mm] or smaller, 30 [mm] or smaller, or 20 [mm] or smaller. The radius of the spiral may mean a distance from the axis A to the center of a cross section of the channel.

The outer peripheral wall 125 may be porous, for example, and may particularly include a porous membrane. Examples of a material of the porous membrane forming the outer peripheral wall 125 may include polycarbonate, for example. Such material is preferable because this suppresses adsorption of biocomponents to the outer peripheral wall.

A mean hole diameter of the porous membrane may be appropriately selected by those skilled in the art according to a size of the components (for example, bioparticles) to be transferred to the outside of the channel 120, and may be, for example, 20 μm or smaller, particularly 15 μm or smaller, more particularly 12 μm or smaller, and still more particularly about 10 μm. The mean hole diameter may be, for example, 1 μm or larger, 3 μm or larger, or 5 μm or larger. Such mean hole diameter is suitable, for example, for removing RBCs from blood by the present technology. The mean hole diameter may be measured using, for example, a confocal microscope. The mean hole diameter may be measured using, for example, a non-contact three-dimensional measurement device to which a principle of the confocal microscope is applied. Examples of the device include an NH series device of Mitaka Kohki Co., Ltd., for example.

The outer peripheral wall 125 may include a porous membrane and a support supporting the membrane. A shape of the channel may be more stably maintained by the support. For example, the support may be arranged so as to wrap the channel 120, or may be arranged so as to cover only a portion of the outer peripheral wall 125. A material of the support is preferably formed so as not to hinder the transfer of the at least part of components to the outside of the channel, and may be, for example, a mesh-shaped material. The material of the support may be, for example, nylon, a polyester-based resin, a polyethylene-based resin, a polypropylene-based resin, a fluorine-based resin, or metal. A mesh opening of the mesh of the support may be set so as not to hinder the transfer of the components to be transferred to the outside of the channel 120, and may be, for example, 10 μm or larger, 15 μm or larger, 20 μm or larger, 25 μm or larger, or 30μ or larger. Furthermore, in order to maintain the channel shape, this may be, for example, 1,000 μm or smaller, 700 μm or smaller, 500 μm or smaller, 400 μm or smaller, or 300 μm or smaller.

Preferably, the outer peripheral wall 125 is formed to allow part of the bioparticles contained in the bioparticle-containing liquid to pass and do not allow the remaining bioparticles to pass. Therefore, part of the bioparticles contained in the liquid may be removed; for example, red blood cells may be removed from the blood.

A shape of a cross section (a plane perpendicular to a proceeding direction of the bioparticle-containing liquid) of the channel 120 may be, for example, circular as illustrated in FIG. 4, but is not limited thereto. The shape may be, for example, elliptical, rectangular, or polygonal other than rectangular. Note that “circular” includes “substantially circular”, and “elliptical” includes “substantially elliptical”. “Rectangular” may be, for example, square or rectangular.

As for a size of the cross section of the channel 120, in a case where the shape of the cross section is circular or elliptical, a diameter or a long diameter may be, for example, 1 mm or larger, 2 mm or larger, or 3 mm or larger. The diameter or long diameter may be, for example, 30 mm or smaller, 20 mm or smaller, or 10 mm or smaller.

As for the size of the cross section of the channel 120, in a case where the shape of the cross section is square or rectangular, one side or a long side may be, for example, 1 mm or larger, 2 mm or larger, or 3 mm or larger. The diameter or long diameter may be, for example, 30 mm or smaller, 20 mm or smaller, or 10 mm or smaller.

The container 110 includes a first inlet 122 that introduces the bioparticle-containing liquid into the channel 120, and a first outlet 124 that discharges the bioparticle-containing liquid that passes through the channel 120 out of the container 110.

The first inlet 122 may be present on a wall surface of the container 110, and may mean, for example, a connection between an introduction channel 121 that introduces the bioparticle-containing liquid from the outside of the container 110 into the container 110 and the channel 120 of the container 110.

The first outlet 124 may be present on the wall surface of the container 110, and may mean a connection between the channel 120 of the container 110 and a discharge channel 123 that discharges the bioparticle-containing liquid from the inside of the container 110 out of the container 110.

Furthermore, the container 110 includes a second inlet 112 that introduces a liquid that receives the components transferred to the outside of the channel 120 into the container 110, and a second outlet 114 that discharges the liquid out of the container 110.

The second inlet 112 may be present on the wall surface of the container 110, and may mean, for example, a supply port that introduces the liquid that receives the components from the outside of the container 110 into the container 110.

The second outlet 114 may be present on the wall surface of the container 110, and may mean, for example, a discharge port that discharges the liquid that receives the components from the inside of the container 110 out of the container 110.

Preferably, the sample preparation device 100 may be formed such that the liquid introduced from the second inlet 112 swirls to flow in the container 110. In order to form such a swirling flow, for example, the second inlet 112 and/or the second outlet 114 may open toward a position deviated from the central axis A of the container.

In order to form the swirling flow, a channel 111 may be connected to the container 110 such that the channel 111 and an outer wall of the container 110 form an acute angle (an angle of, for example, smaller than 90°, particularly equal to or smaller than 80°, more particularly equal to or smaller than) 70° at a connection between the channel 111 and the outer wall of the container 110, for example. For example, the second inlet 112 may be provided so that the liquid immediately after being introduced from the second inlet 112 does not proceed to the center of the container 110. More specifically, the second inlet 112 may be provided so that the liquid immediately after being introduced from the second inlet flows toward a portion between the central axis of the container 110 and a container inner wall surface.

Furthermore, in order to form the swirling flow, a channel 113 may be connected to the container 110 such that the channel 113 and an outer wall of the container 110 form an acute angle (an angle of, for example, smaller than 90°, particularly equal to or smaller than 80°, more particularly equal to or smaller than 70°) at a connection between the channel 113 and the outer wall of the container 110, for example.

Preferably, the second inlet 112 and the second outlet 114 may be arranged in different positions in a sedimentation direction (for example, a gravity action direction) of the bioparticles. Preferably, the second inlet 112 is arranged behind in the sedimentation direction, and the second outlet 114 is arranged ahead in the sedimentation direction. For example, the second inlet 112 may be arranged above the second outlet 114 in the gravity action direction. Therefore, the components (particularly, the bioparticles) transferred to the outside of the channel 120 may be efficiently discharged from the second outlet 114.

Preferably, the first inlet 122 and the first outlet 124 may be arranged in different positions in a sedimentation direction (for example, a gravity action direction) of the bioparticles. Preferably, the first inlet 122 is arranged behind (on a side from which sedimentation occurs) in the sedimentation direction, and the first outlet 124 is arranged ahead (on a side to which the sedimentation occurs) in the sedimentation direction. For example, the first inlet 122 may be arranged above the first outlet 124 in the gravity action direction. Therefore, the bioparticle-containing liquid is urged to proceed from the first inlet 122 to the first outlet 124 in the channel 120.

In FIG. 4, one second inlet 112 that introduces the liquid that receives the components transferred to the outside of the channel 120 into the container 110 is provided, but the number of second inlets is not limited to one and may be plural. For example, two, three, or four second inlets may be connected to the container 110.

Furthermore, in FIG. 4, one second outlet 114 that discharges the liquid introduced from the second inlet 112 out of the container 110 is provided, but the number of second outlets 114 is not limited to one and may be plural. For example, two, three, or four second outlets may be connected to the container 110.

In this manner, in the present technology, the container may include a plurality of the second inlets and a plurality of the second outlets.

(2-2) Example of System Including Sample Preparation Device and Example of Sample Preparation Method

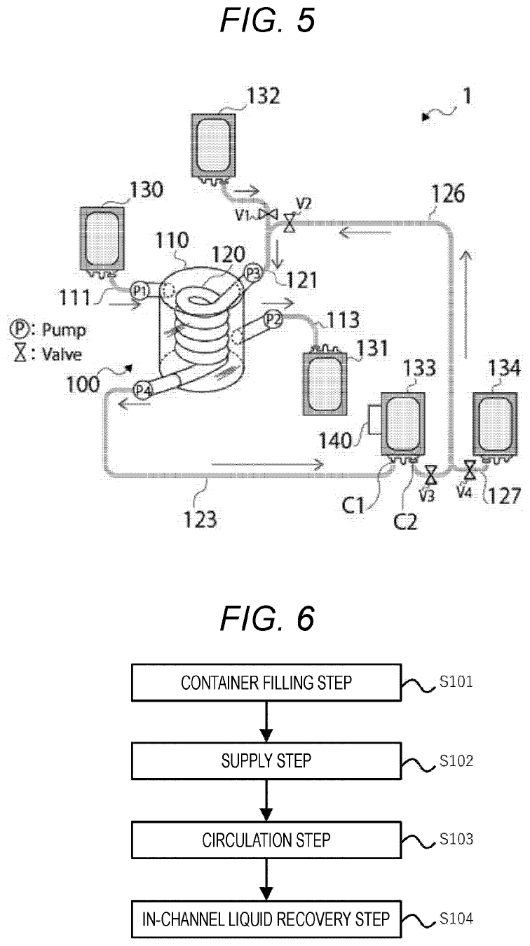

In order to prepare a sample using the sample preparation device according to the present technology, for example, a sample preparation system as illustrated in FIG. 5 may be formed. Hereinafter, the sample preparation system will be described, and next, an example of a sample preparation method using the system will be described.

(2-2-1) Configuration Example of Sample Preparation System

A sample preparation system 1 of FIG. 5 includes the sample preparation device 100 described with reference to FIG. 4. A configuration of the channel connected to the sample preparation device 100 and the container containing various liquids will be hereinafter described.

A pump P1 is provided on the channel 111 that introduces the liquid (the liquid that receives the components transferred to the outside of the channel 120) into the container 110 of the sample preparation device 100. Moreover, the channel 111 is connected to a container 130 in which the liquid that receives the components transferred to the outside of the channel 120 is stored. By driving the pump P1, the liquid in the container 130 is supplied to the container 110.

A pump P2 is provided on the channel 113 that introduces the liquid (the liquid that receives the components transferred to the outside of the channel 120) from the container 110 of the sample preparation device 100. Moreover, the channel 113 is connected to a recovery container (also referred to as a “waste liquid container”) 131 that recovers the discharged liquid. By driving the pump P2, the liquid in the container 110 is recovered into the waste liquid container 131.

A pump P3 is provided on the channel 121 that introduces the bioparticle-containing liquid into the channel 120 of the sample preparation device. Moreover, the channel 121 is connected to a container 132 in which the bioparticle-containing liquid is stored. By driving the pump P3, the bioparticle-containing liquid in the container 132 is supplied to the channel 120.

Furthermore, a valve V1 is provided on the channel 121. By opening and closing the valve V1, the supply of the bioparticle-containing liquid in the container 132 to the channel 120 becomes possible or impossible.

A pump P4 is provided on the channel 123 that discharges the bioparticle-containing liquid that passes through the channel 120 of the sample preparation device from the container 110. Moreover, the channel 123 is connected to a recovery container 133 into which the bioparticle-containing liquid that passes through the channel 120 is recovered. By driving the pump P4, the bioparticle-containing liquid that passes through the channel 120 is delivered to the recovery container 133.

Furthermore, a valve V1 is provided on the channel 121. By opening and closing the valve V1, the supply of the bioparticle-containing liquid in the container 132 to the channel 120 becomes possible or impossible.

The recovery container 133 is provided with connectors C1 and C2. The connector C1 connects the channel 123 to the recovery container 133. The connector C2 connects the container 133 to a channel 126 provided with a valve V3. By opening and closing of the valve V3, it becomes possible or impossible to allow the liquid in the recovery container 133 to proceed to the channel 120 through the channel 126.

The channel 126 is formed to join the channel 121. On the channel 126, a valve V2 is provided immediately before a junction of the channel 126 and the channel 121. By opening and closing the valve V2, it becomes possible or impossible to allow the liquid in the recovery container 133 or a container 134 to proceed to the channel 120 through the channel 126.

A channel 127 connected to the container 134 filled with a solution for recovery joins the channel 126. A valve V4 is provided on the channel 127. By opening and closing the valve V4, it becomes possible or impossible to allow the liquid in the recovery container 134 to proceed to the channel 120 through the channel 126.

The sample preparation system 1 further includes an analysis device 140 that analyzes the liquid in the recovery container 133. The analysis device 140 may be, for example, a device that analyzes a color of the liquid in the recovery container 133, a device that measures a concentration of the components contained in the liquid, or a device that measures a content of the bioparticles contained in the liquid.

Note that the analysis device 140 may be formed as an analysis device that analyzes the liquid flowing in the channel 123 or 126 instead of analyzing the liquid in the container 133.

(2-2-2) Sample Preparation Method

A flowchart of a sample preparation method by the sample preparation system 1 is illustrated in FIG. 6. As illustrated in FIG. 6, the sample preparation method by the sample preparation system 1 includes container filling step S101 of filling the container 110 with the liquid that receives the components transferred to the outside of the channel 120, supply step S102 of supplying bioparticle-containing liquid to the channel 120, circulation step S103 of allowing the bioparticle-containing liquid to circulate through a circulation channel including the channel 120, and in-channel liquid recovery step S104.

At container filling step S101, the container 110 (particularly, a space occupied by the channel 120 is excluded from the space in the container 110) of the sample preparation device 100 is filled with the liquid in the container 130. As described above, the liquid in the container 130 is the liquid that receives the components transferred to the outside of the channel 120 when the bioparticle-containing liquid is allowed to flow in the channel 120 through the outer peripheral wall of the channel 120.

In order to perform container filling step S101, the pump P1 is driven. By driving the pump P1, the liquid in the container 130 is introduced into the container 110 through the channel 111.

At supply step S102, the bioparticle-containing liquid contained in the container 132 is supplied to the channel 120.

In order to perform supply step S102, the valve V1 is opened, and then the pumps P3 and P4 are driven. By driving the pumps P3 and P4, the bioparticle-containing liquid in the container 132 passes through the channel 121 and then introduced into the channel 120. Since the channel 120 has a spiral shape, a centrifugal force acts on the bioparticle-containing liquid flowing in the channel 120. The centrifugal force acts so as to allow the bioparticles contained in the bioparticle-containing liquid to proceed toward the outer peripheral wall of the channel 120. Therefore, part of components (for example, part of bioparticles) of the bioparticle-containing liquid are transferred to the outside of the channel 120 through the outer peripheral wall of the channel 120.

The components transferred to the outside of the channel 120 are received by the liquid filling the container 110. The liquid containing the components is recovered into the waste liquid container 131. In order to perform the recovery, the pump P2 is driven. Therefore, the liquid containing the components proceeds to the waste liquid container 131 through the channel 113.

At circulation step S103, the bioparticle-containing liquid is allowed to circulate through the circulation channel including the channel 120. The circulation channel may be formed such that the bioparticle-containing liquid output from the channel 120 from the first outlet 124 is supplied to the channel 120 again from the first inlet 122. For example, in the sample preparation system illustrated in FIG. 5, the circulation channel includes the channel 120, the channel 123, the container 133, the channel 126, and a portion from the junction with the channel 126 to the first inlet 122 of the channel 121.

Prior to circulation step S103, first, it is possible that entire bioparticle-containing liquid in the container 132 is supplied to the channel 120, so that the supply is finished, or a predetermined amount of the bioparticle-containing liquid in the container 132 is supplied to the channel 120, so that the supply is finished. When the supply is finished, the valve V1 is closed.

Then, in order to perform circulation step S103, the valves V2 and V3 are opened, and the pumps P3 and P4 are driven. By driving the pumps P3 and P4, the bioparticle-containing liquid in the circulation channel is allowed to circulate in the circulation channel, and thus repeatedly passes through the channel 120.

Therefore, as described regarding supply step S102, part of components (for example, part of bioparticles) of the bioparticle-containing liquid are transferred to the outside of the channel 120 through the outer peripheral wall of the channel 120. That is, more components may be removed from the bioparticle-containing liquid.

Furthermore, a concentration and a content ratio of the components in the bioparticle-containing liquid may be adjusted by controlling a time for performing circulation step S103.

In this manner, in the present technology, it may be preferably formed such that the bioparticle-containing liquid output from the first outlet may enter the container again from the first inlet.

The components transferred to the outside of the channel 120 are received by the liquid filling the container 110. The liquid containing the components is recovered into the waste liquid container 131. In order to perform the recovery, the pump P2 is driven. Therefore, the liquid containing the components proceeds to the waste liquid container 131 through the channel 113.

A finishing timing of circulation step S103 may be appropriately selected by a user. For example, the user may observe the bioparticle-containing liquid in the recovery container 133, and the user may determine the finishing timing of circulation step S103, or the user may determine the finishing timing of circulation step S103 according to an analysis result of the liquid by the analysis device 140. For example, the valve V3 may be closed to finish circulation step S103.

Furthermore, the driving of the pumps P3 and P4 may be stopped for this finishing.

Circulation step S103 may be automatically finished. For example, circulation step S103 may be automatically finished in response to acquisition of a predetermined analysis result by the analysis device 140. For example, the valve V3 may be closed in response to acquisition of a predetermined analysis result.

Note that, in a case where a desired sample is obtained by allowing the bioparticle-containing liquid to pass through the channel 120 once, it is possible that circulation step S103 is not performed.

In the present technology, the bioparticle-containing liquid present in the recovery container 133 when supply step S102 is finished or when circulation step S103 is finished may be used as the prepared sample.

In a preferred embodiment of the present technology, in-channel liquid recovery step S104 of recovering the bioparticle-containing liquid present in the channel into the recovery container 133 may be executed after supply step S102 is finished or after circulation step S103 is finished. Therefore, more samples may be recovered into the recovery container 133.

At in-channel liquid recovery step S104, for example, the bioparticle-containing liquid present in a portion other than the recovery container 133 (the channel 120, the channel 123, the channel 126, and a portion from the junction with the channel 126 to the first inlet 122 of the channel 121) in the circulation channel is recovered into the recovery container 133.

In order to perform in-channel liquid recovery step S104, the pumps P3 and P4 are driven in a state in which the valves V2 and V4 are opened. By the driving, the solution for recovery in the container 134 is supplied to the channel 126 through the channel 127, and thereafter flows in the channels 121, 120, and 123. When the solution for recovery flows in this manner, the bioparticle-containing liquid present in these channels is recovered into the recovery container 133. After the recovery, all the pumps are stopped, and in-channel liquid recovery step S104 is finished. Then, the bioparticle-containing liquid in the recovery container 133 may be handled as the sample prepared by the sample preparation system 1.

Note that, those skilled in the art may know in advance a volume in the channel in which the solution for recovery flows, so that they may appropriately determine the finishing timing of in-channel liquid recovery step S104.

The present technology also provides a sample preparation method. The sample preparation method includes, for example, the supply step. Moreover, the sample preparation method may further include the circulation step and/or the in-channel liquid recovery step.

(2-2-3) Operation Example (Example of Preparing Sample Having High Content Ratio of White Blood Cells (WBCs) by Removing Red Blood Cells (RBC) from Blood)

Hereinafter, an operation example for preparing a sample having a high content ratio of white blood cells (WBCs) by removing red blood cells (RBCs) from blood by the sample preparation system 1 will be described.

Prior to container filling step S101, the container 132 is filled with the blood and the container 134 is filled with a liquid for recovery. Furthermore, the container 130 is filled with a liquid (for example, a buffer and the like) that receives the RBCs output from the channel 120 as the blood flows in the channel 120. Hereinafter, the liquid that receives the RBCs is also referred to as a cleaning liquid.

At container filling step S101, the pump P1 is driven. By driving the pump P1, the cleaning liquid in the container 130 is introduced into the container 110 through the channel 111. Therefore, the container 110 (particularly, a space occupied by the channel 120 is excluded from a space in the container 110) is filled with the cleaning liquid.

At container filling step S101, for example, it is possible that other pumps are not driven. Furthermore, at container filling step S101, all the valves V1 to V4 may be closed.

At supply step S102, the valve V1 is opened, and then the pumps P3 and P4 are driven. By driving the pumps P3 and P4, the blood in the container 132 passes through the channel 121 and then introduced into the channel 120. Since the channel 120 has a spiral shape, a centrifugal force acts on the bioparticle-containing liquid flowing in the channel 120. Then, due to the action of the centrifugal force, the RBCs are transferred to the outside of the channel 120 through the outer peripheral wall of the channel 120.

The RBCs are received by the cleaning liquid filling the container 110. Then, the cleaning liquid containing the RBCs is recovered into the waste liquid container 131. In order to perform the recovery, the pump P2 is driven. Therefore, the cleaning liquid containing the RBCs proceeds to the waste liquid container 131 through the channel 113. In order to perform the recovery, the pump P1 may be driven. By driving the pumps P1 and P2, a swirling flow may be generated in the container 110. Therefore, for example, it is possible to efficiently recover the waste liquid. For example, the RBCs may be prevented from settling in the container.

When the entire blood in the container 132 is supplied to the channel 120, the valve V1 is closed.

In order to perform circulation step S103, the valves V2 and V3 are opened. At circulation step S103, the pumps P3 and P4 are driven in a state in which the valves V2 and V3 are opened. By driving the pumps P3 and P4, the blood circulates through the circulation channel (the channel 120, the channel 123, the container 133, the channel 126, and a portion from the junction with the channel 126 to the first inlet 122 of the channel 121), whereby the blood repeatedly passes through the channel 120. Therefore, more RBCs are transferred to the outside of the channel 120 through the outer peripheral wall of the channel 120, and are received by the cleaning liquid. That is, the red blood cells are removed from the blood in the circulation channel.

The cleaning liquid that receives the RBCs is recovered into the waste liquid container 131. In order to perform the recovery, the pump P2 is driven. Therefore, the cleaning liquid proceeds to the waste liquid container 131 through the channel 113.

As circulation step S103 is continued, the RBCs are gradually removed from the blood in the circulation channel. That is, the ratio of the WBCs in the blood cells is increased. Therefore, redness of the liquid in the container 133 decreases over time at circulation step S103. For example, when the redness in the container 133 disappears (that is, when an RBC concentration is sufficiently lowered), the valve V3 is closed. In this manner, a blood sample in which the content ratio of the RBCs is decreased and the content ratio of the WBCs is increased is obtained in the container 133.

A closing timing of the valve V3 may be determined by the user observing the redness.

Alternatively, the liquid may be monitored in real time by an analysis device (for example, a concentration sensor that measures a concentration or a color sensor that detects a color) that analyzes the liquid in any position in the circulation channel. Then, the valve V3 may be closed at a stage at which a predetermined analysis result is obtained (for example, at a stage at which a predetermined concentration or color is obtained).

At in-channel liquid recovery step S104, the pumps P3 and P4 are driven in a state in which the valves V2 and V4 are opened. By the driving, the solution for recovery in the container 134 is supplied to the channel 126 through the channel 127, and thereafter flows in the channels 121, 120, and 123. When the solution for recovery flows in this manner, the blood sample (blood sample in which the RBC content decreases) present in these channels is recovered into the recovery container 133. After the recovery, all the pumps are stopped, and in-channel liquid recovery step S104 is finished. Then, the blood sample in the recovery container 133 is handled as the sample prepared by the sample preparation system 1.

(3) First Variation of Sample Preparation Device According to Present Technology (Channel Having Cylindrical Shape)

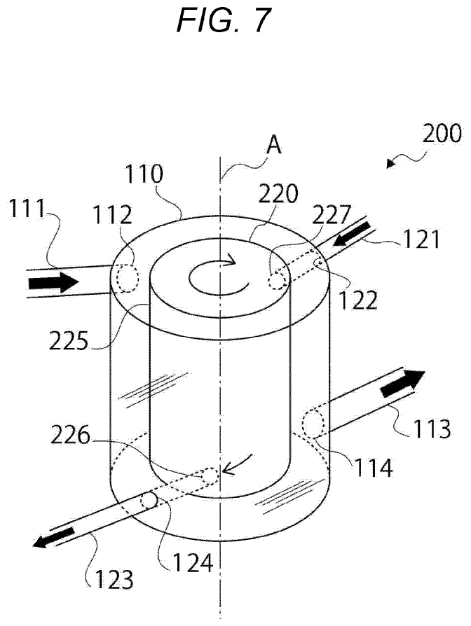

A shape of a channel formed such that a centrifugal force acts included in a sample preparation device according to the present technology is not limited to the spiral shape described in (2) above, and may be, for example, a cylindrical shape. The sample preparation device having the channel in the cylindrical shape will be hereinafter described with reference to FIG. 7.

A sample preparation device 200 illustrated in FIG. 7 is the same as the sample preparation device 100 described in (2) above with reference to FIG. 4 except that this includes a channel 220 in a cylindrical shape in place of the channel 120 in a spiral shape. Therefore, a container 110 and various channels are as described in (2) above, and the description thereof also applies to the sample preparation device 200 of FIG. 7. Hereinafter, the channel 220 in the cylindrical shape is described.

As illustrated in FIG. 7, the channel 220 has the cylindrical shape. When the bioparticle-containing liquid flows in the channel having the cylindrical shape, particularly, when this flows so as to swirl around an axis A (flows so as to form a vortex) as indicated by an arrow in FIG. 7, a centrifugal force acts on the bioparticle-containing liquid. By the centrifugal force, at least one component (for example, a bioparticle) contained in the liquid is transferred to the outside of the channel 220 through an outer peripheral wall 225 of the channel 220. The transferred component is received by a liquid in the container 110.

In this manner, in the present technology, the sample preparation device may be formed such that the bioparticle-containing liquid forms a flow that goes around the axis of the cylindrical shape.

In this specification, the cylindrical shape includes a straight cylindrical shape and an oblique cylindrical shape. As illustrated in FIG. 7, the channel 220 preferably has the straight spiral shape.

Dimensions of two bottom surfaces forming the cylindrical shape may be the same as or different from each other. For example, an upper bottom surface (bottom surface on a side from which sedimentation occurs in a sedimentation direction of the bioparticles) may be larger or smaller than a lower bottom surface (bottom surface on a side to which the sedimentation occurs in the sedimentation direction).

The description regarding the outer peripheral wall 125 in (2) above applies to the outer peripheral wall 225 of the channel 220. For example, the outer peripheral wall 225 may be porous as described in (2) above.

Furthermore, the channel 220 in the cylindrical shape is provided with a third inlet 227 for introducing the bioparticle-containing liquid, and a third outlet 226 that discharges the bioparticle-containing liquid that flows in the channel 220. The third inlet 227 and/or the third outlet 226 may be formed to form the swirling flow as described above in the channel 220 in the cylindrical shape. In order to form such swirling flow, for example, the third inlet 227 and/or the third outlet 226 may open toward a position deviated from the central axis A of the container.

In order to form the swirling flow, a channel 121 may be connected to the outer peripheral wall 225 such that the channel 121 and an outer wall of the container 220 form an acute angle (an angle of, for example, smaller than 90°, particularly equal to or smaller than 80°, more particularly equal to or smaller than 70°) at a connection between the channel 121 and the outer peripheral wall 225 of the channel 220, for example. For example, the third inlet 227 may be provided so that the bioparticle-containing liquid immediately after being introduced from the third inlet 227 does not proceed to the center of the channel 220. More specifically, the third inlet 227 may be provided so that the bioparticle-containing liquid immediately after being introduced from the third inlet 227 flows toward a portion between the central axis of the channel 220 and a container inner wall surface.

Furthermore, in order to form the swirling flow, a channel 123 may be connected to the outer peripheral wall 225 of the channel 220 such that the channel 123 and the outer peripheral wall 225 of the channel 220 form an acute angle (an angle of, for example, smaller than 90°, particularly equal to or smaller than 80°, more particularly equal to or smaller than 70°) at a connection between the channel 123 and the outer peripheral wall 225 of the channel 220, for example.

Preferably, the third inlet 227 and the third outlet 226 may be arranged in different positions in the sedimentation direction (for example, a gravity action direction) of the bioparticles. Preferably, the third inlet 227 is arranged behind (on a side from which sedimentation occurs) in the sedimentation direction, and the third outlet 226 is arranged ahead (on a side to which the sedimentation occurs) in the sedimentation direction. For example, the third inlet 227 may be arranged above the third outlet 226 in the gravity action direction. Therefore, the bioparticle-containing liquid swirling in the channel 220 is urged to proceed from the third inlet 227 to the third outlet 226, and the bioparticle-containing liquid is efficiently discharged from the third outlet 226.

(4) Second Variation of Sample Preparation Device According to Present Technology (Channel Having U Shape)

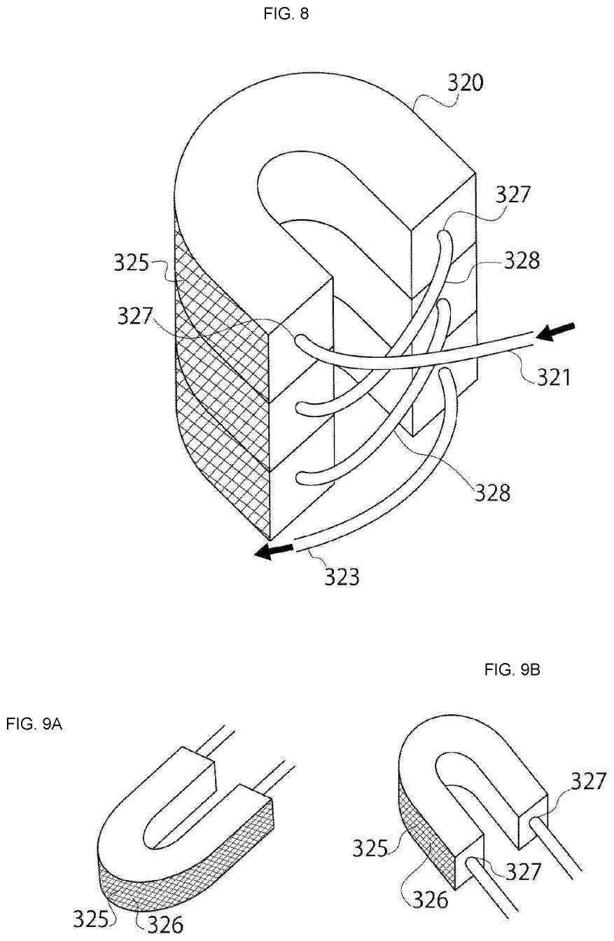

A shape of a channel formed such that a centrifugal force acts included in a sample preparation device according to the present technology is not limited to the spiral shape described in (2) above and the cylindrical shape described in (3) above, and may be, for example, a U shape. An example of the channel in the U shape will be hereinafter described with reference to FIGS. 8 and 9.

A channel 320 illustrated in FIG. 8 is obtained by stacking a plurality of U-shaped channel units illustrated in FIGS. 9A and 9B. In the U-shaped channel unit illustrated in FIGS. 9A and 9B, an outer peripheral wall on which a centrifugal force acts is formed such that at least part of components of a bioparticle-containing liquid may be transferred to the outside of the channel. For example, this includes a porous membrane 326 and a support 325 supporting the membrane. The membrane 326 and the support 325 are as described in (2) above. By combining a plurality of such U-shaped channel units, particularly, by stacking a plurality of them as illustrated in FIG. 8, a function similar to that of the channel in the spiral shape described in (2) above is exhibited. As illustrated in FIG. 8, respective U-shaped channel units are connected by a channel 328 such as a tube, for example.

For example, regarding the channel 320 illustrated in FIG. 8, as indicated by an upper arrow, the bioparticle-containing liquid is introduced from a channel 321, and then the liquid enters the U-shaped channel unit from an inlet 327-1 of the U-shaped channel unit. Then, the bioparticle-containing liquid is output from an outlet 327-2 of the U-shaped channel unit, passes through the tube 328, and enters again the U-shaped channel unit immediately below. By repeating this, at least part of components (for example, part of bioparticles) contained in the bioparticle-containing liquid are transferred to the outside of the channel from the outer peripheral wall.

In this manner, the sample preparation device of the present technology includes a plurality of the channels having the U-shape, and the plurality of U-shaped channels may be connected to each other to form a single line of flow.

(5) Third Variation of Sample Preparation Device According to Present Technology (Connection of Plurality of Devices)

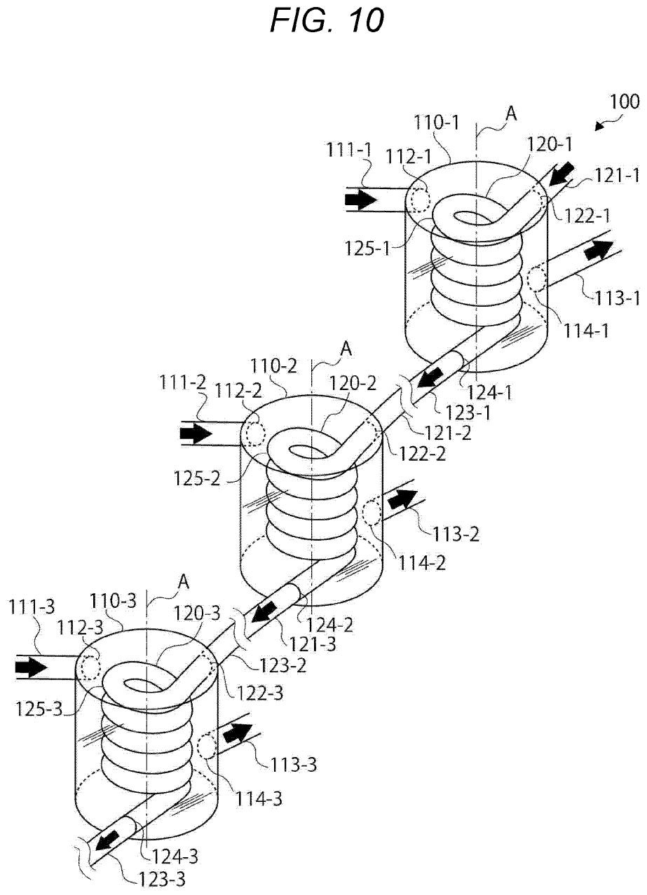

A sample preparation device according to the present technology may include a plurality of sets of the container and the channel formed such that a centrifugal force acts. In the sample preparation device including a plurality of the sets, for example, sizes of components that may be transferred to the outside from an outer peripheral wall of the channel of respective sets may be different from each other. More specifically, the outer peripheral wall of the channel of each set may be porous, and hole sizes of the outer peripheral wall of the channel of the respective sets may be different from each other. A plurality of types of components (particularly, bioparticles) having different sizes may be fractionated by the sample preparation device including the plurality of sets. The variation is hereinafter described with reference to FIG. 10.

A sample preparation device 1000 illustrated in FIG. 10 includes three sets of the container 110 and channel 120 described in (2) above. Specifically, a container 110-1 and a channel 120-1 (hereinafter, also referred to as a “first set”), a container 110-2 and a channel 120-2 (hereinafter, also referred to as a “second set”), and a container 110-3 and a channel 120-3 (hereinafter, also referred to as a “third set”) are included.

A discharge channel 123-1 of the first set is connected to an introduction channel 121-2 of the second set. Therefore, a bioparticle-containing liquid that passes through the channel 120-1 in the first set is introduced into the channel 120-2 in the second set.

A discharge channel 123-2 of the second set is connected to an introduction channel 121-3 of the third set. Therefore, the bioparticle-containing liquid that passes through the channel 120-2 in the second set is introduced into the channel 120-3 in the third set.

As described above, by connecting a plurality of sets in series, the components that should be separated from the bioparticle-containing liquid may be efficiently separated.

Furthermore, in a case where a plurality of sets is directly made in this manner, components (particularly, bioparticles) having different sizes may be fractionated. By forming in this manner, a hole diameter of an outer peripheral wall 125-1 of the first set is made smaller than a hole diameter of an outer peripheral wall 125-2 of the second set, and the hole diameter of the outer peripheral wall 125-2 of the second set is made smaller than a hole diameter of an outer peripheral wall 125-3 of the third set. That is, the hole diameter of the outer peripheral wall is made larger in a direction in which the bioparticle-containing liquid flows. Therefore, for example, bioparticles having the smallest size are discharged from a discharge channel 113-1 of the first set, bioparticles having the second smallest size are discharged from a discharge channel 113-2 of the second set, and then bioparticles having the third smallest size are discharged from a discharge channel 113-3 of the third set. Then, from a discharge channel 123-3 of the third set, bioparticles having a size larger than that of these three types of bioparticles are discharged. In this manner, four types of particles having different sizes may be fractionated.

In this manner, the sample preparation device of the present technology may include a plurality of sets of the container and the channel, and the sizes of the components that may be transferred to the outside from the outer peripheral wall of the channel of the respective sets may be different from each other. For example, the sample preparation device of the present technology may include a plurality of sets of the container and the channel, the outer peripheral wall of the channel of each set may be porous, and the hole sizes of the outer peripheral wall of the channel of the respective sets may be different from each other.

(6) Fourth Variation of Sample Preparation Device According to Present Technology (Density Adjustment)

In a sample preparation device according to the present technology, a channel may be further provided in a channel formed such that a centrifugal force acts on a bioparticle-containing liquid.

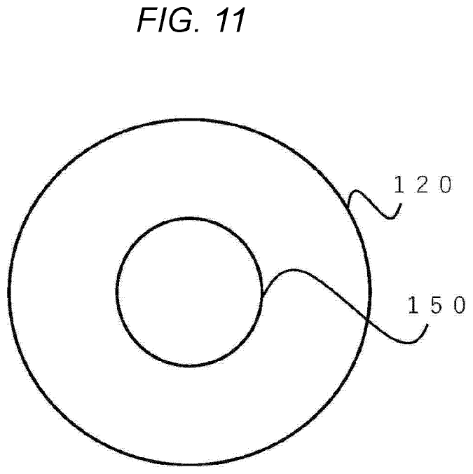

FIG. 11 illustrates a schematic diagram of a cross section of such channel. As illustrated in FIG. 11, a channel 150 (hereinafter also referred to as an “inner channel”) may be provided in a channel 120. The inner channel 150 may be formed to allow a liquid to flow on an inner side thereof (inside a circle represented by reference numeral 150), and may preferably be formed to be able to adjust a pressure in the inner channel 150 by the liquid. In the channel 120, the bioparticle-containing liquid flows in a space between a circle represented by reference numeral 120 and the circle represented by reference numeral 150.

By allowing the liquid to flow in the inner channel 150 to adjust the pressure of the inner channel, the liquid is supplied from the inner channel 150 to the channel 120. Therefore, a concentration in the channel 120 decreases, and concentration adjustment (dilution) may be performed.

The channel 150 may include, for example, a membrane filter, and may particularly include a membrane filter having a hole diameter smaller than a size of bioparticles that are wanted to be recovered. That is, the hole diameter of the channel 150 is preferably smaller than a hole diameter of the outer peripheral wall 125.

### 2. Second Embodiment (Sample Preparation System)

The present technology also provides a sample preparation system including the sample preparation device described in 1. above. The sample preparation system may be formed as described in (2) of 1. above, for example.

For example, the sample preparation system may include at least one pump that supplies a bioparticle-containing liquid to a channel formed such that a centrifugal force acts on the bioparticle-containing liquid flowing in the channel. The at least one pump may be formed as P3 described in (2) of 1. above, for example.

The sample preparation system may include at least one pump that discharges the bioparticle-containing liquid from the channel formed such that the centrifugal force acts on the bioparticle-containing liquid flowing in the channel. The at least one pump may be formed as P4 described in (2) of 1. above, for example.

For example, the sample preparation system may include at least one pump that supplies a liquid that receives components transferred to the outside of a channel 120 to a container in which the channel is accommodated. The at least one pump may be formed as P1 described in (2) of 1. above, for example.

For example, the sample preparation system may include at least one pump that discharges the liquid that receives the components transferred to the outside of the channel 120 from the container in which the channel is accommodated. The at least one pump may be formed as P2 described in (2) of 1. above, for example.

Furthermore, the sample preparation system of the present technology may include at least one valve provided on a channel that supplies the bioparticle-containing liquid to the channel formed such that a centrifugal force acts on the liquid. The at least one valve may control the supply, and particularly, the supply may be made possible or impossible by opening and closing the valve. The at least one valve may be formed as V1 described in (2) of 1. above, for example.

Furthermore, the sample preparation system of the present technology may be formed such that the bioparticle-containing liquid output from the first outlet may enter the container again from the first inlet. For example, the sample preparation system of the present technology may include a circulation channel that allows the bioparticle-containing liquid output from the first outlet to enter the container again from the first inlet. The circulation channel may be formed as described in (2) of 1. above. A recovery container into which a sample (bioparticle-containing liquid) prepared by the sample preparation system of the present technology is recovered may be provided on the circulation channel.

Furthermore, the sample preparation system of the present technology may include an analysis device that analyzes a liquid in the channel or a liquid in the container forming the system.

The analysis device may be provided, for example, in any position on the circulation channel, and may be, for example, an analysis device that analyzes the bioparticle-containing liquid that passes through the channel formed such that the centrifugal force acts described in (2) of 1.

Furthermore, the analysis device may be an analysis device that analyzes the liquid that receives the components transferred to the outside from the channel formed such that a centrifugal force acts. The analysis device may be, for example, an analysis device that analyzes the liquid in the container 110 described in (2) of 1. above, or may be an analysis device that analyzes the liquid flowing in the channel 113 or the liquid in the container 131.

The analysis device may be a concentration measurement device that measures a concentration of components contained in a liquid, or may be a color measurement device that measures a color of a liquid. An operation of the sample preparation system may be controlled according to an analysis result by the analysis device, particularly according to a measurement result of the concentration or color, and various processes (for example, circulation step described in (2) of 1. above and the like) by the sample preparation system may be started or finished, for example.

The sample preparation system of the present technology may further include a control unit that controls an operation of each element forming the system. The control unit may control, for example, an operation of the pump group and/or the valve group described above. For example, the control unit may control the operation of the pump group and/or the valve group according to a predetermined program.

Furthermore, the control unit may be formed to receive the analysis result by the analysis device. The control unit may control the operation of the pump group and/or the valve group according to the analysis result by the analysis device. For example, the control unit may control driving of any one or two or more of the pump groups in response to reception of a predetermined analysis result, and particularly may start or stop the driving. Furthermore, the control unit may control opening and closing of any one or two or more of the valve groups in response to reception of a predetermined analysis result.

The control unit may be formed as an information processing device (computer), and a function of the control unit may be implemented by, for example, a general-purpose computer.

Note that, the present technology may also have a following configuration.

[1]

A sample preparation device including:
a container; and
a channel in which a bioparticle-containing liquid flows, the channel accommodated in the container, in which
the channel is formed such that a centrifugal force acts on the bioparticle-containing liquid, and
an outer peripheral wall of the channel is formed such that at least part of components of the bioparticle-containing liquid may be transferred to an outside of the channel.
   [2]

The sample preparation device according to [1], in which

the channel has a spiral shape.

[3]

The sample preparation device according to [2], in which

the channel has a curved shape so as to go around one axis.

[4]

The sample preparation device according to [3], in which

the channel is formed so as to go around the axis one or more times.

[5]

The sample preparation device according to any one of [1] to [3], in which

the outer peripheral wall of the channel has a predetermined curvature.

[6]

The sample preparation device according to [1], in which

the channel has a cylindrical shape.

[7]

The sample preparation device according to [6], formed such that the bioparticle-containing liquid forms a flow that goes around the axis of the cylindrical shape.

[8]

The sample preparation device according to [1], in which

the channel has a U shape.

[9]

The sample preparation device according to [8], including:

a plurality of the channels having a U-shape, the plurality of U-shaped channels connected to each other to form a single line of flow.

[10]

The sample preparation device according to any one of [1] to [9], in which

the outer peripheral wall is porous.

[11]

The sample preparation device according to any one of [1] to [10], in which

the outer peripheral wall allows part of bioparticles contained in the bioparticle-containing liquid to pass and does not allow remaining bioparticles to pass.

[12]

The sample preparation device according to any one of [1] to [11], in which
the container includes a first inlet that introduces the bioparticle-containing liquid into the channel, and a first outlet that discharges the bioparticle-containing liquid that passes through the channel to an outside of the container, and
the container includes a second inlet that introduces a liquid that receives the components transferred to the outside of the channel into the container, and a second outlet that discharges the liquid to the outside of the container.
   [13]

The sample preparation device according to [12], formed such that the liquid introduced from the second inlet swirls to flow in the container.

[14]

The sample preparation device according to [12] or [13], in which

the second inlet and the second outlet open toward a position deviated from a central axis of the container.

[15]

The sample preparation device according to any one of [12] to [14], in which

the second inlet is arranged above the second outlet.

[16]

The sample preparation device according to any one of [12] to [15], in which

the container includes a plurality of the second inlets and a plurality of the second outlets.

[17]

The sample preparation device according to any one of [1] to [16], including:
a plurality of sets of the container and the channel, in which
sizes of components that may be transferred to the outside from the outer peripheral wall of the channel of respective sets are different from each other.
   [18]

The sample preparation device according to any one of [12] to [17], formed such that the bioparticle-containing liquid output from the first outlet may enter the container again from the first inlet.

[19]

The sample preparation device according to any one of [1] to [18], used for separating blood components.

[20]

A sample preparation system including:
a sample preparation device including a container, and a channel in which a bioparticle-containing liquid flows, the channel accommodated in the container, the channel formed such that a centrifugal force acts on the bioparticle-containing liquid, and an outer peripheral wall of the channel formed such that components of the bioparticle-containing liquid may be transferred to an outside of the channel; and
an analysis device that executes analysis of the bioparticle-containing liquid that passes through the channel.
   [21]

A sample preparation device including:
a container; and
a channel in which a microparticle-containing liquid flows, the channel accommodated in the container, in which
the channel is formed such that a centrifugal force acts on the microparticle-containing liquid, and
an outer peripheral wall of the channel is formed such that at least part of components of the microparticle-containing liquid may be transferred to an outside of the channel.
   [22]

A sample preparation system including:
a sample preparation device including a container, and a channel in which a microparticle-containing liquid flows, the channel accommodated in the container, the channel formed such that a centrifugal force acts on the microparticle-containing liquid, and an outer peripheral wall of the channel formed such that components of the microparticle-containing liquid may be transferred to an outside of the channel; and
an analysis device that executes analysis of the microparticle-containing liquid that passes through the channel.

### REFERENCE SIGNS LIST

100 Sample preparation device
110 Container
120 Channel
125 Outer peripheral wall

1. A sample preparation device comprising:
a container; and
a channel in which a bioparticle-containing liquid flows, the channel accommodated in the container, wherein
the channel is formed such that a centrifugal force acts on the bioparticle-containing liquid, and
an outer peripheral wall of the channel is formed such that at least part of components of the bioparticle-containing liquid may be transferred to an outside of the channel.

2. The sample preparation device according to claim 1, wherein
the channel has a spiral shape.

3. The sample preparation device according to claim 2, wherein
the channel has a curved shape so as to go around one axis.

4. The sample preparation device according to claim 3, wherein
the channel is formed so as to go around the axis one or more times.

5. The sample preparation device according to claim 1, wherein
the outer peripheral wall of the channel has a predetermined curvature.

6. The sample preparation device according to claim 1, wherein
the channel has a cylindrical shape.

7. The sample preparation device according to claim 6, formed such that the bioparticle-containing liquid forms a flow that goes around the axis of the cylindrical shape.

8. The sample preparation device according to claim 1, wherein
the channel has a U shape.

9. The sample preparation device according to claim 8, comprising:
a plurality of the channels having a U-shape, the plurality of U-shaped channels connected to each other to form a single line of flow.

10. The sample preparation device according to claim 1, wherein
the outer peripheral wall is porous.

11. The sample preparation device according to claim 1, wherein
the outer peripheral wall allows part of bioparticles contained in the bioparticle-containing liquid to pass and does not allow remaining bioparticles to pass.

12. The sample preparation device according to claim 1, wherein
the container includes a first inlet that introduces the bioparticle-containing liquid into the channel, and a first outlet that discharges the bioparticle-containing liquid that passes through the channel to an outside of the container, and
the container includes a second inlet that introduces a liquid that receives the components transferred to the outside of the channel into the container, and a second outlet that discharges the liquid to the outside of the container.

13. The sample preparation device according to claim 12, formed such that the liquid introduced from the second inlet swirls to flow in the container.

14. The sample preparation device according to claim 12, wherein
the second inlet and the second outlet open toward a position deviated from a central axis of the container.

15. The sample preparation device according to claim 12, wherein
the second inlet is arranged above the second outlet.

16. The sample preparation device according to claim 12, wherein
the container includes a plurality of the second inlets and a plurality of the second outlets.

17. The sample preparation device according to claim 1, comprising:
a plurality of sets of the container and the channel, wherein
sizes of components that may be transferred to the outside from the outer peripheral wall of the channel of respective sets are different from each other.

18. The sample preparation device according to claim 12, formed such that the bioparticle-containing liquid output from the first outlet may enter the container again from the first inlet.

19. The sample preparation device according to claim 1, used for separating blood components.

20. A sample preparation system comprising:
a sample preparation device including a container, and a channel in which a bioparticle-containing liquid flows, the channel accommodated in the container, the channel formed such that a centrifugal force acts on the bioparticle-containing liquid, and an outer peripheral wall of the channel formed such that components of the bioparticle-containing liquid may be transferred to an outside of the channel; and
an analysis device that executes analysis of the bioparticle-containing liquid that passes through the channel.

21. A sample preparation device comprising:
a container; and
a channel in which a microparticle-containing liquid flows, the channel accommodated in the container, wherein
the channel is formed such that a centrifugal force acts on the microparticle-containing liquid, and
an outer peripheral wall of the channel is formed such that at least part of components of the microparticle-containing liquid may be transferred to an outside of the channel.

22. A sample preparation system comprising:
a sample preparation device including a container, and a channel in which a microparticle-containing liquid flows, the channel accommodated in the container, the channel formed such that a centrifugal force acts on the microparticle-containing liquid, and an outer peripheral wall of the channel formed such that components of the microparticle-containing liquid may be transferred to an outside of the channel; and
an analysis device that executes analysis of the microparticle-containing liquid that passes through the channel.
